(19) United States
(12) Patent Application Publication
(10) Pub. No.: US 2021/0215693 A1
(43) Pub. Date: Jul. 15, 2021

(54) Method and System for Identifying Human Individuals Infected with COVID-19 as Being at High Risk of Progression to Severe or Critical Disease

(71) Applicant: Roni Amiel, Orlando, FL (US)

(72) Inventor: Roni Amiel, Orlando, FL (US)

(21) Appl. No.: 16/890,678

(22) Filed: Jun. 2, 2020

Related U.S. Application Data
(63) Continuation-in-part of application No. 16/743,195, filed on Jan. 15, 2020.

(51) Int. Cl. G01N 33/569; G16H 50/30; G16B 5/00; G06N 3/02

(52) U.S. Cl. CPC G01N 33/569; G16H 50/30; G16B 5/00; G01N 2333/91188; G01N 2800/60; G01N 2333/775; G01N 2333/4713; G06N 3/02

(57) ABSTRACT

Identifying in the presence of an SARS-CoV-2, infection of COVID-19 in a human subject and/or for classifying the risk of such infection progressing to a severe or critical disease, using a system functionally associated with at least one analyzer for analyzing the blood sample, is disclosed. The system includes at least one of an input interface or a transceiver, and one or more processors functionally associated therewith. A storage medium associated with the processor(s) has stored instructions to receive biographical information relating to the human subject, and instructions to receive measurements of a plurality of serum biomarkers. Further stored are instructions to apply a neural network algorithm to the measurements of the plurality of biomarkers and the biographical information, and instructions to identify, based on an output of the neural network algorithm, a COVID-19 infection or to classify the risk of such infection progressing to a severe or critical disease.

### FIELD OF THE DISCLOSED TECHNOLOGY

The disclosed technology relates generally to methods and systems for diagnosing COVID-19 (SARS-CoV2 infection-related disease) in a human subject, and, more specifically, to methods and systems (in-vitro diagnostics) for identifying that a human subject, infected with COVID-19, is at high risk of progression to severe or critical disease, using a blood sample.

### BACKGROUND OF THE DISCLOSED TECHNOLOGY

A pandemic of a new disease, Coronavirus, also known as COVID-19 and SARS-CoV2, began at the end of 2019 in China and rapidly spread throughout the world. By the middle of May, more than 4.5 Million people worldwide had been infected, and more than 300,000 died.

During the pandemic it was noticed that the vast majority of patients show mild symptoms, similar to that of an intense flu infection, while a small percentage of patients—typically ranging from 1% of infected patients to 10% of infected patients—suffered severe symptoms, required hospitalization, artificial respiration support, and some ultimately died. However, statistical analysis of those suffering from severe symptoms of COVID-19 shows that approximately 40% of adults are at a higher risk for developing a severe case of COVID-19, either due to age or due to underlying medical conditions as obesity and diabetes mellitus.

Several relatively large-scale case studies have reported the clinical features of patients with coronavirus COVID-19. Laboratory medicine plays an essential role in the early detection, diagnosis and management of many diseases. Coronavirus COVID-19 makes no exception to this rule. The role of laboratory diagnostics extends far beyond etiological diagnosis and epidemiologic surveillance, whereby in vitro diagnostic tests are commonly used for assessing disease severity, for defining the prognosis, for following-up of patients, for guiding treatment and for their therapeutic monitoring. The currently available data suggests that many laboratory parameters are disturbed in patients with coronavirus COVID-19 and some of these may also be considered significant predictors of adverse clinical outcomes.

Early identification of people at high risk for developing a severe case of COVID-19 would be beneficial both for taking precautionary measures with respect to those people (e.g. stricter social distancing practices, using preventive or preparatory medications, and the like) and for ensuring that medical staff and medical facilities are properly and sufficiently equipped to treat patients when the disease becomes severe.

There is thus a need in the art for a system and/or a method to evaluate whether a human subject is at high risk of COVID-19 progressing into a severe disease, where the evaluation may be carried out following infection of the subject or prior thereto.

### SUMMARY OF THE DISCLOSED TECHNOLOGY

The present disclosure relates to a method and a system for non-invasively and accurately identifying the presence of a liver condition in a human subject, and classifying the severity of such a liver condition.

In accordance with an embodiment of the disclosed technology, there is provided a method for identifying in the presence of SARS-CoV-2 infection of COVID-19 disease in a human subject and/or for classifying the risk of such infection progressing to a severe or critical disease. In accordance with the method, biographical information relating to the human subject is obtained. The biographical information includes at least age, gender, height, and weight. A plurality of analyzers are used to obtain, from serum or plasma in a blood sample obtained from the human subject, measurements of a plurality of serum biomarkers. The plurality of serum biomarkers include at least two biomarkers selected from the group consisting of lymphocyte count, ferritin, D-dimer, LDH (lactate dehydrogenase), and C reactive protein. Using a processor executing instructions stored in a non-transitory computer memory, a neural network algorithm is applied to the measurements of the plurality of biomarkers and the biographical information. Based on an output of the neural network algorithm, in the presence of SARS-CoV-2 infection, COVID-19 disease is identified and/or a risk of such infection progressing to a severe or critical disease is classified.

In some embodiments, the obtained measurements further include a measurement of at least one biomarker selected from the group consisting of Alpha-2-Macroglobulin, Apolipoprotein A1, Haptoglobin, total Bilirubin, gamma-glutamyl transpeptidase (GGT), alanine-aminotransferase (ALT), aspartate aminotransferase (AST), Total fasting cholesterol, fasting triglycerides, and fasting glucose.

In some embodiments, prior to the application of the neural network algorithm, at least some of the measurements of the plurality of serum biomarkers or at least one data item of the biographical information (personal medical history) are pre-processed.

In some embodiments, the pre-processing includes standardizing at least some of the measurements of the plurality of serum biomarkers and at least one data item of the biographical information. In some embodiments, the pre-processing includes logarithmically scaling at least some of the measurements of the plurality of serum biomarkers.

In accordance with another embodiment of the disclosed technology, there is provided a system for identifying in the presence of an SARS-CoV-2 infection of COVID-19 disease in a human subject and/or for classifying the risk of such infection progressing to a severe or critical disease, the system being functionally associated with at least one analyzer for analyzing the blood sample. The system includes at least one of an input interface or a transceiver, one or more processors functionally associated with the at least one input interface or transceiver, and a non-transitory computer readable storage medium for instructions execution by the one or more processors. The non-transitory computer readable storage medium has stored instructions to receive biographical information relating to the human subject, including at least age, gender, height, and weight. The storage medium further has stored instructions to receive, from the at least one analyzer, measurements of a plurality of serum biomarkers, the plurality of serum biomarkers including at least two biomarkers selected from the group consisting of lymphocyte count, ferritin, D-dimer, LDH (lactate dehydrogenase), and C reactive protein. Further stored instructions are to apply a neural network algorithm to the measurements of the plurality of biomarkers and the biographical information. The storage medium has stored additional instructions to identify, based on an output of said neural network algorithm, the presence of the infection of COVID-19 in the human subject or to classify, based on the output of the neural network algorithm, the risk of such infection progressing to a severe or critical disease.

In some embodiments, the instructions to receive measurements include instructions to additionally receive a measurement of at least one biomarker selected from the group consisting of Alpha-2-Macroglobulin, Apolipoprotein A1, Haptoglobin, total Bilirubin, gamma-glutamyl transpeptidase (GGT), alanine-aminotransferase (ALT), aspartate aminotransferase (AST), Total fasting cholesterol, fasting triglycerides, and fasting glucose.

In some embodiments, the non-transitory computer readable storage medium further has stored instructions, to be executed prior to execution of the instructions to applying the neural network algorithm, to pre-process at least some of the measurements of the plurality of serum biomarkers or at least one data item of the biographical information.

In some embodiments, the instructions to pre-process include instructions to logarithmically scale at least some of the measurements of the plurality of serum biomarkers.

In some embodiments, the instructions to pre-process include instructions to standardize at least some of the measurements of the plurality of serum biomarkers and at least one data item of the biographical information.

### BRIEF DESCRIPTION OF THE DRAWINGS

FIG. 1 is a schematic illustration of a system for identifying in the presence of SARS-CoV-2 infection, COVID-19 and/or classifying the likelihood or risk of progression of the disease in a specific subject to a severe or critical case, according to an embodiment of the disclosed technology.

FIG. 2 is a flow-chart of a method for identifying the presence of COVID-19, and/or classifying the likelihood or risk of progression of the disease in a specific subject to a severe or critical case, according to an embodiment of the disclosed technology.

FIG. 3 shows a high-level block diagram of a device that may be used to carry out the disclosed technology.

### DETAILED DESCRIPTION OF EMBODIMENTS OF THE DISCLOSED TECHNOLOGY

In an embodiment of the disclosed technology, in the presence of an infection of SARS-CoV-2, COVID-19 in a human subject may be identified and/or the risk of such infection progressing to a severe or critical disease may be classified, using a system functionally associated with at least one analyzer for analyzing the blood sample, is disclosed. The system includes at least one of an input interface or a transceiver, and one or more processors functionally associated therewith. A storage medium associated with the processor(s) has stored instructions to receive biographical information relating to the human subject, and instructions to receive measurements of a plurality of serum biomarkers. Further stored are instructions to apply a neural network algorithm to the measurements of the plurality of biomarkers and the biographical information, and instructions to identify, based on an output of the neural network algorithm, a COVID-19 due to SARS-CoV-2 infection or to classify the risk of such disease progressing to a severe or critical disease.

Embodiments of the disclosed technology will become clearer in view of the following description of the drawings.

In the context of the present specification and claims, the term “approximately” is defined as being within 10% of a target number or measure.

It should be understood that the use of “and/or” is defined inclusively such that the term “a and/or b” should be read to include the sets: “a and b,” “a or b,” “a,” “b.”

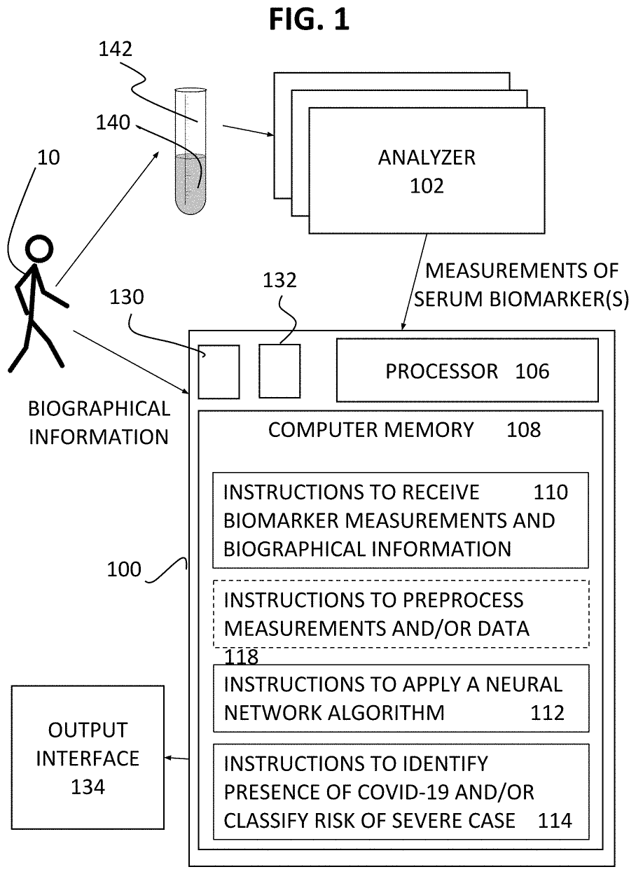

Reference is now made to FIG. 1, which is a schematic illustration of a system for identifying the presence of COVID-19, and/or classifying the likelihood or risk of progression of the disease in a specific subject to a severe or critical case, according to an embodiment of the disclosed technology. As seen, a system 100 according to the disclosed technology comprises a computing device, functionally associated with a plurality of analyzers 102. The system 100 includes one or more processors 106, and a non-transitory computer readable storage medium 108, which stores instructions to be executed by the processor(s) 106.

Computer readable storage medium 108 has stored:
instructions 110 to receive, from analyzers 102, measurements of serum biomarkers obtained from blood of a human subject 10, as well as biographical information of the human subject 10;
instructions 112 to apply a neural network algorithm to the measurements of serum biomarkers and to the biographical information of the human subject; and
instructions 114 to identify, based on the output of the neural network algorithm, the presence or absence of infection with COVID-19, and/or the likelihood of an infection with COVID-19 to progress to a severe or critical case.

In some embodiments, computer readable storage medium 108 further has stored instructions 118 to scale at least some of the measurements of serum biomarkers, and/or instructions to standardize at least some of the measurements of serum biomarkers and/or at least some of the biographical information.

The instructions 110 to receive measurements of serum biomarkers include instructions to receive measurements of at least two, at least three, or all of the following biomarkers:
Lymphocyte count; Lymphocytes are white cells that are implied in the in-nate immune response to viral infections including SARS-CoV-2. Lymphopenia was one of the most frequent abnormalities in Covid-19 (35-75% of cases) (Lippi G. et al. CCLM. 2020). 85% of critically ill patients with COVID-19 showed lymphopenia (Huang C. et al. Lancet 2020, Yang X. et al. The Lancet Respiratory Medicine. 2020) The presence of lymphopenia is a signature of severe COVID-19, ICU patients suffering this infection had a median lymphocyte count of 800 cells/mm, with non-survivors exhibiting persistent lymphopenia. (Wang D. et al. JAMA 2020) Early recognition of lymphopenia could be useful to assist prompt recognition of severe patients who require or will shortly require critical care (Bermejo-Martin J F. Et al. Journal of Infection. 2020, Allenbach Y. et al. medRxiv. 2020) In patients with diabetes mellitus, a major comorbidity for COVID-19 severity, lymphopenia was associated with increased odds ratios for severe Covid-19 (tracheal intubation for mechanical ventilation) and/or death within 7 days of admission. (Cariou B. et al. Diabetologia 2020)
Ferritin; Ferritin is an iron storage protein found in large quantities at the intracellular level (in the liver and white blood cells). It is correlated to the amount of iron present in the blood where ferritin is present transiently. Ferritin blood level could rise with inflammation and, therefore, high levels of blood Ferritin is also regarded as a predictor of fatality in COVID-19, suggesting that mortality might be due to virally driven hyperinflammation. (Chen G. et al. J Clin Invest 2020, Mehta P. et al. The Lancet 2020, Wu C. et al. JAMA Intern Med 2020) 25% of patients with severe respiratory failure have secondary macrophage activating syndrome (MAS), that could properly be classified by using ferritin. (Bornstein S R Lancet Diabetes Endocrinol. 2020)
D-Dimer; D-dimers are a product of the breakdown of fibrin (the final element of blood clotting) during the fibrinolysis process. Studies identified a positive correlation with mortality for D-Dimers in patients with Covid-19, being associated with ICU transfer and/or death within 14 days from admission. Patients having worst outcome or death have almost two-fold levels of D-Dimers compared to patients that are ICU-free or alive (Allenbach Y. et al. medRxiv 2020) D-dimer may be an important predictor of disease severity related to coagulopathy along with fibrinogen and platelets in patients with COVID-19. (Zhou et al. medRxiv 2020)
Lactate deshydrogenase (LDH); This enzyme is normally found in most body tissues, and only in small amounts in the blood. When tissues are damaged, cells release LDH causing its concentration in the blood to in-crease. Significantly elevated levels of LDH were observed in non-survivors compared with survivors suggesting massive hemolysis and tissue damage throughout the clinical course deterioration. (Zhou F. et al. The Lancet. 2020, Cariou B. et al. Diabetologia 2020)
C Reactive Protein (CRP). CRP is an acute phase protein synthesized mainly by the liver but also by adipose tissue. It plays an important role in inflammatory reactions, mainly induced by bacterial infection. In the early stage of COVID-19 CRP levels were positively correlated with lung lesions and could reflect disease severity (Wang L. Med Mal Infect 2020) CRP level were independently associated with a worse prognosis in several studies. (Zhang C. Lancet Gastroenterology & Hepatology. 2020, Cheng Y. et al. Kidney International. 2020, Lo I L, Int J Biol Sci. 2020). A CRP level increment of 1.63 per 100 mg/L was associated with an increased risk of ICU requirement or death (Allenbach Y. et al. medRxiv 2020)

The instructions 110 to receive measurements of serum biomarkers include instructions to receive at least the human subject's age, gender, height, and weight.

In some embodiments, the instructions 110 to receive measurements of serum biomarkers include instructions to additionally receive measurements of liver disease serum biomarkers, for example as described in U.S. patent application Ser. No. 16/743,195 filed Jan. 15, 2020, which is incorporated by reference as if fully set forth herein. In some such embodiments, the instructions are to additionally receive measurement(s) of at least some of:
Alpha-2-Macroglobulin;
Apolipoprotein A1;
Haptoglobin;
total Bilirubin;
gamma-glutamyl transpeptidase (GGT);
alanine-aminotransferase (ALT);
aspartate aminotransferase (AST);
Total fasting cholesterol;
fasting triglycerides; and
fasting glucose.

In some embodiments, system 100 further includes, or is functionally associated with, at least one input interface 130, such as a keyboard, touchscreen, touchpad, mouse, and the like, functionally associated with processor 106. A user, such as a medical practitioner, may use the input interface(s) 130 to provide at least some of the data received when executing instructions 110, such as at least some of the biographical information.

In some embodiments, system 100 further includes at least one transceiver 132, functionally associated with processor 106, and adapted for communication with the analyzer(s) 102 and/or with other devices adapted to provide the inputs received by execution of instructions 110, such as a scale.

In some embodiments, instructions 114 to identify the presence or absence of infection with COVID-19 and/or the likelihood of an infection with COVID-19 to progress to a severe or critical case, further include instructions to provide an output including the identified presence and/or likelihood. In some such embodiments, system 100 further includes, or is functionally associated with, an output interface 134, such as a display screen or a speaker, via which the output is visually or audibly provided to a user, such as a medical practitioner. In some embodiments, the output may be provided in an electronic communication, such as an e-mail message, for example via transceiver 132.

Typically, a blood sample 140, used to extract the measurements of the serum biomarkers received system 100, is collected from the human subject using test tubes 142 as known in the art. In some embodiments, the blood sample must have sufficient volume to provide a minimum 500 microliter volume of plasma or serum, after centrifugation thereof. In some embodiments, the blood sample must have sufficient volume to provide a 200 microliter volume of plasma or serum, after centrifugation thereof.

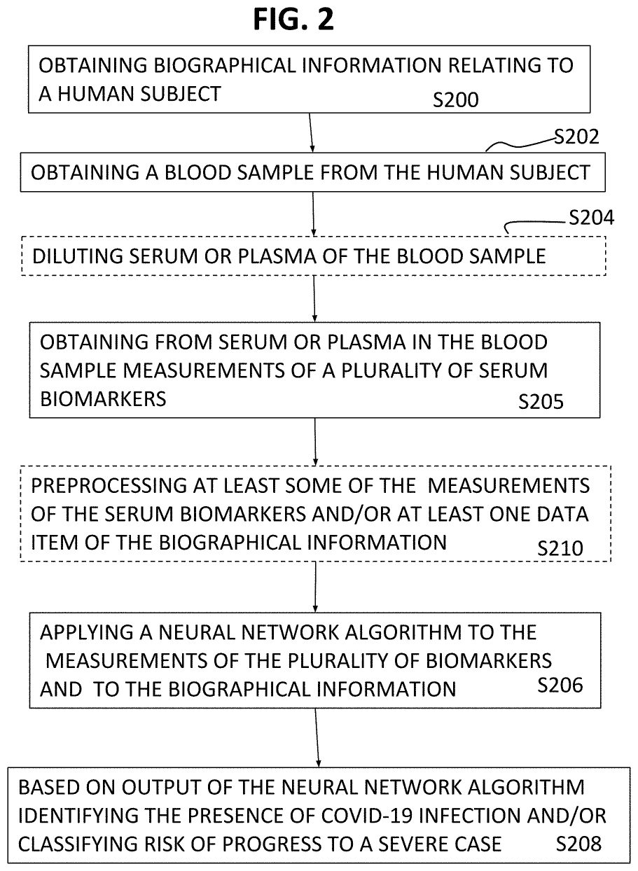

Reference is now additionally made to FIG. 2, which is a flow-chart of a method for identifying the presence of COVID-19, and/or classifying the likelihood or risk of progression of the disease in a specific subject to a severe or critical case, according to an embodiment of the disclosed technology. The method of FIG. 2 is described herein with respect to the system of FIG. 1. However, any other suitable system may be used to implement the method of FIG. 2.

As seen in FIG. 2, at step S200, biographical information relating to a human subject, such as subject 10 of FIG. 1, is obtained, for example by processor 106 executing instructions 110. In some embodiments, the biographical information includes at least the subject's age, gender, height, and weight. At step S202, which may occur before, after, or concurrently with step S200, a blood sample of the human subject is obtained. As discussed hereinabove with respect to FIG. 1, the blood sample is typically obtained in test tubes, using methods known in the art. Serum or plasma is then extracted from the blood sample, using methods known in the art, such as centrifugation.

In some embodiments, the obtained blood sample may optionally be diluted, or otherwise preprocessed, at step S204. This may occur when the blood sample includes components which would interfere with the remainder of the method, such as lipids.

At step S205, measurements of a plurality of serum biomarkers are obtained from the serum or plasma in the blood sample, for example by processor 106 executing instructions 110. The measurements are computed by one or more analyzers, such as analyzers 102 of FIG. 1, and are received from the analyzer(s). The obtained measurements include measurements for at least two, at least three, and preferably all of:
Lymphocyte count;
Ferritin;
D-Dimer;
LDH; and
C Reactive Protein.

In some embodiments, the obtained measurements further include at least some of:
Alpha-2-Macroglobulin;
Apolipoprotein A1;
Haptoglobin;
total Bilirubin;
gamma-glutamyl transpeptidase (GGT);
alanine-aminotransferase (ALT);
aspartate aminotransferase (AST);
Total fasting cholesterol;
fasting triglycerides; and
fasting glucose.

At step S206, a neural network algorithm is applied to at least some of, or to all of, the measurements of the plurality of biomarkers and to at least some of, or to all of, the obtained biographical information, for example by processor 106 executing instructions 112. Based on the output of the neural network algorithm, at step S208, in the presence of infection with SARS-CoV-2, severity of COVID-19 is identified. Additionally, or alternatively, at step S208, classifying the likelihood or risk of progression of the disease in the specific subject, if/when the subject is infected, to a severe or critical case is classified, for example by processor 106 executing instructions 114.

In some embodiments, prior to application of the neural network algorithm at step S206, at least some of the serum biomarker measurements and/or some biographical information data items preprocessed, for example by processor 106 executing instructions 118. In some such embodiments, the values used for the neural network algorithm, at step S206, are the preprocessed values resulting from step S210. In some such embodiments, the preprocessing includes logarithmically scaling at least some of the serum biomarker measurements, for example using logarithmic base 10. In some embodiments, the preprocessing includes standardizing values of at least some of the serum biomarker measurements and/or of at least some biographical information data items.

In some embodiments, in which the preprocessing includes logarithmic scaling of serum biomarker measurements. In some embodiments, in which the preprocessing includes standardizing of serum biomarker measurements and/or of biographical information data items.

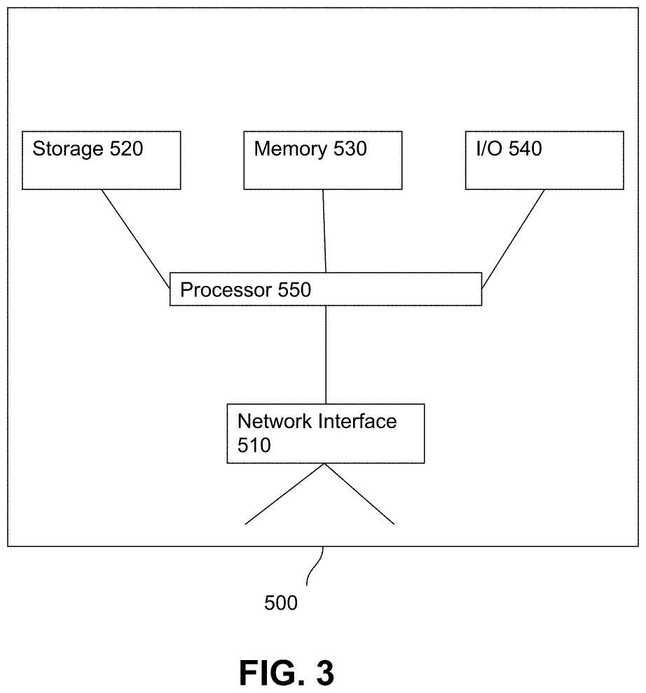

FIG. 3 shows a high-level block diagram of a device that may be used to carry out the disclosed technology. Device 500 comprises a processor 550 that controls the overall operation of the computer by executing the device's program instructions which define such operation. The device's program instructions may be stored in a storage device 520 (e.g., magnetic disk, database) and loaded into memory 530 when execution of the console's program instructions is desired. Thus, the device's operation will be defined by the device's program instructions stored in memory 530 and/or storage 520, and the console will be controlled by processor 550 executing the console's program instructions. A device 500 also includes one or a plurality of input network interfaces for communicating with other devices via a network (e.g., the internet). The device 500 further includes an electrical input interface. A device 500 also includes one or more output network interfaces 510 for communicating with other devices. Device 500 also includes input/output 540 representing devices which allow for user interaction with a computer (e.g., display, keyboard, mouse, speakers, buttons, etc.). One skilled in the art will recognize that an implementation of an actual device will contain other components as well, and that FIG. 3 is a high level representation of some of the components of such a device for illustrative purposes. It should also be understood by one skilled in the art that the method and devices depicted in FIGS. 1 and 2 may be implemented using a device such as is shown in FIG. 3.

For purposes of this disclosure, the term “substantially” is defined as “at least 95% of” the term which it modifies.

Any device or aspect of the technology can “comprise” or “consist of” the item it modifies, whether explicitly written as such or otherwise.

When the term “or” is used, it creates a group which has within either term being connected by the conjunction as well as both terms being connected by the conjunction.

While the disclosed technology has been taught with specific reference to the above embodiments, a person having ordinary skill in the art will recognize that changes can be made in form and detail without departing from the spirit and the scope of the disclosed technology. The described embodiments are to be considered in all respects only as illustrative and not restrictive. All changes that come within the meaning and range of equivalency of the claims are to be embraced within their scope. Combinations of any of the methods and apparatuses described hereinabove are also contemplated and within the scope of the invention.

1. A method for identifying in the presence of an infection SARS-CoV-2, COVID-19 in a human subject and/or for classifying the risk of such infection progressing to a severe or critical disease, the method comprising:
obtaining biographical information relating to the human subject, said biographical information including at least age, gender, height, and weight;
using a plurality of analyzers, obtaining from serum or plasma in a blood sample obtained from the human subject measurements of a plurality of serum biomarkers, said plurality of serum biomarkers including at least two biomarkers selected from the group consisting of lymphocyte count, ferritin, D-dimer, LDH (lactate dehydrogenase), and C reactive protein;
using a processor executing instructions stored in a non-transitory computer memory, applying a neural network algorithm to said measurements of said plurality of biomarkers and said biographical information; and
based on an output of said neural network algorithm, identifying the presence of the infection of COVID-19 in the human subject or classifying the risk of such infection progressing to a severe or critical disease.

2. The method of claim 1, wherein said obtaining said measurements of said plurality of serum biomarkers further includes additionally obtaining a measurement of at least one biomarker selected from the group consisting of Alpha-2-Macroglobulin, Apolipoprotein A1, Haptoglobin, total Bilirubin, gamma-glutamyl transpeptidase (GGT), alanine-aminotransferase (ALT), aspartate aminotransferase (AST), Total fasting cholesterol, fasting triglycerides, and fasting glucose.

3. The method of claim 1, further comprising, prior to said applying said neural network algorithm, pre-processing at least some of said measurements of said plurality of serum biomarkers or at least one data item of said biographical information.

4. The method of claim 3, wherein said pre-processing comprises logarithmically scaling at least some of said measurements of said plurality of serum biomarkers.

5. The method of claim 3, wherein said pre-processing comprises standardizing at least some of said measurements of said plurality of serum biomarkers and at least one data item of said biographical information.

6. A system for identifying in the presence of an infection of SARS-CoV-2, COVID-19 in a human subject and/or for classifying the risk of such infection progressing to a severe or critical disease, the system being functionally associated with at least one analyzer for analyzing the blood sample, the system comprising:
at least one of an input interface or a transceiver;
one or more processors functionally associated with said at least one input interface or transceiver; and
a non-transitory computer readable storage medium for instructions execution by the one or more processors, the non-transitory computer readable storage medium having stored:
instructions to receive biographical information relating to the human subject, said biographical information including at least age, gender, height, and weight;
instructions to receive, from said at least one analyzer, measurements of a plurality of serum biomarkers, said plurality of serum biomarkers including at least two biomarkers selected from the group consisting of lymphocyte count, ferritin, D-dimer, LDH (lactate dehydrogenase), and C reactive protein;
instructions to apply a neural network algorithm to said measurements of said plurality of biomarkers and said biographical information; and
instructions to identify, based on an output of said neural network algorithm, in the presence of the infection of SARS-CoV-2, COVID-19 in the human subject or to classify, based on said output of said neural network algorithm, the risk of such infection progressing to a severe or critical disease.

7. The system of claim 6, wherein said instructions to receive measurements of a plurality of serum biomarkers further include instructions to additionally receive measurements of at least one biomarker selected from the group consisting of Alpha-2-Macroglobulin, Apolipoprotein A1, Haptoglobin, total Bilirubin, gamma-glutamyl transpeptidase (GGT), alanine-aminotransferase (ALT), aspartate aminotransferase (AST), Total fasting cholesterol, fasting triglycerides, and fasting glucose.

8. The system of claim 6, wherein said non-transitory computer readable storage medium further has stored instructions, to be executed prior to execution of said instructions to applying said neural network algorithm, to pre-process at least some of said measurements of said plurality of serum biomarkers or at least one data item of said biographical information.

9. The system of claim 8, wherein said instructions to pre-process comprise instructions to logarithmically scale at least some of said measurements of said plurality of serum biomarkers.

10. The system of claim 9, wherein said instructions to pre-process comprise instructions to standardize at least some of said measurements of said plurality of serum biomarkers and at least one data item of said biographical information.
